(19) United States
(12) Patent Application Publication
(10) Pub. No.: US 2006/0014714 A1
(43) Pub. Date: Jan. 19, 2006

(54) Genetic induction of anti-viral immune response and genetic vaccine for viruses

(76) Inventors: Harriet L. Robinson, Southborough, MA (US); Joel R. Haynes, Middleton, WI (US)

Correspondence Address: FOLEY AND LARDNER LLP;SUITE 500, 3000 K STREET NW, WASHINGTON, DC, 20007, US

(21) Appl. No.: 11/178,588

(22) Filed: Jul. 12, 2005

Related U.S. Application Data
(63) Continuation of application No. 08/208,394, filed on Mar. 9, 1994, now abandoned.
Application No. 08/208,394 is a continuation-in-part of application No. 08/103,024, filed on Aug. 4, 1993, now abandoned.
Application No. 08/103,024 is a continuation-in-part of application No. 07/850,189, filed on Mar. 11, 1992, now abandoned.
(63) Continuation-in-part of application No. 08/009,833, filed on Jan. 27, 1993, now Pat. No. 5,643,578.
Application No. 08/009,833 is a continuation-in-part of application No. 07/855,562, filed on Mar. 23, 1992, now abandoned.

(51) Int. Cl. A61K 48/00; A61K 39/145

(52) U.S. Cl. 514/44; 424/209.1

(57) ABSTRACT

An approach to genetic vaccine methodology is described. A genetic construction encoding antigenic determinants of a virus is transfected into cells of the vaccinated individuals using a particle acceleration protocol so as to express the viral antigens in healthy cells to produce an immune response to those antigens.

### RELATED APPLICATION

This application is a continuation-in-part of U.S. Ser. No. 08/103,024 filed Aug. 4, 1993, which was a continuation-in-part of U.S. Ser. No. 07/850,189, filed Mar. 11, 1992, the present application also being a continuation-in-part of U.S. Ser. No. 08/009,883 filed Jan. 27, 1993 which was a continuation-in-part of Ser. No. 07/855,562 filed Mar. 23, 1992.

### FIELD OF THE INVENTION

The present invention relates to the general field of genetic vaccines and relates, in particular, to genetic agents delivered into the skin or mucosal tissues of animals to induce immune response, and more particularly to genetic vaccines for viral pathogens delivered into skin or mucosal tissues by particle acceleration.

### BACKGROUND OF THE INVENTION

The vaccination of individuals to render the vaccinated individuals resistant to the development of infectious disease is one of the oldest forms of preventive care in medicine. Previously, vaccines for viral and bacterial pathogens for pediatric, adult, and veterinary usage were derived directly from the infectious organisms and could be categorized as falling into one of three broad categories: live attenuated, killed, and subunit vaccines. Although the three categories of vaccines differ significantly in their development and mode of actions, the administration of any of these three categories of these vaccines is intended to result in production of specific immunological responses to the pathogen, following one or more inoculations of the vaccine. The resulting immunological responses may or may not completely protect the individual against subsequent infection, but will usually prevent the manifestation of disease symptoms and significantly limit the extent of any subsequent infection.

The techniques of modern molecular biology have enabled a variety of new vaccine strategies to be developed which are in various stages of pre-clinical and clinical development. The intent of these efforts is not only to produce new vaccines for old diseases, but also to yield new vaccines for infectious diseases in which classical vaccine development strategies have so far proven unsuccessful. Notably, the recent identification and spread of immunodeficiency viruses is an example of a pathogen for which classical vaccine development strategies have not yielded effective control to date.

The first broad category of classical vaccine is live attenuated vaccines. A live attenuated vaccine represents a specific strain of the pathogenic virus, or bacterium, which has been altered so as to lose its pathogenicity, but not its ability to infect and replicate in humans. Live attenuated vaccines are regarded as the most effective form of vaccine because they establish a true infection within the individual. The replicating pathogen and its infection of human cells stimulates both humoral and cellular compartments of the immune system as well as long-lasting immunological memory. Thus, live attenuated vaccines for viral and intracellular bacterial infections stimulate the production of neutralizing antibodies, as well as cytotoxic T-lymphocytes (CTLs), usually after only a single inoculation.

The ability of live attenuated vaccines to stimulate the production of CTLs is believed to be an important reason for the comparative effectiveness of live attenuated vaccines. CTLs are recognized as the main component of the immune system responsible for the actual clearing of viral and intracellular bacterial infections. CTLs are triggered by the production of foreign proteins in individual infected cells of the hosts, the infected cells processing the antigen and presenting the specific antigenic determinants on the cell surface for immunological recognition.

The induction of CTL immunity by attenuated vaccines is due to the establishment of an actual, though limited, infection in the host cells including the production of foreign antigens in the individual infected cells. The vaccination process resulting from a live attenuated vaccine also results in the induction of immunological memory, which manifests itself in the prompt expansion of specific CTL clones and antibody-producing plasma cells in the event of future exposure to a pathogenic form of the infectious agent, resulting in the rapid clearing of this infection and practical protection from disease.

An important disadvantage of live attenuated vaccines is that they have an inherent tendency to revert to a new virulent phenotype through random genetic mutation. Although statistically such a reversion is a rare event for attenuated viral vaccines in common use today, such vaccines are administered on such a large scale that occasional reversions are inevitable, and documented cases of vaccine-induced illnesses exist. In addition, complications are sometimes observed when attenuated vaccines lead to the establishment of disseminated infections due to a lowered state of immune system competence in the vaccine recipient. Further limitations on the development of attenuated vaccines are that appropriate attenuated strains can be difficult to identify for some pathogens and that the frequency of mutagenic drift for some pathogens can be so great that the risk associated with reversion are simply unacceptable. A virus for which this latter point is particularly well exemplified is the human immunodeficiency virus (HIV) in which the lack of an appropriate animal model, as well as an incomplete understanding of its pathogenic mechanism, makes the identification and testing of attenuated mutant virus strains effectively impossible. Even if such mutants could be identified, the rapid rate of genetic drift and the tendency of retroviruses, such as HIV, to recombine would likely lead to an unacceptable level of instability in any attenuated phenotype of the virus. Due to these complications, the production of a live attenuated vaccine for certain viruses may be unacceptable, even though this approach efficiently produces the desired cytotoxic cellular immunity and immunological memory.

The second category of vaccines consists of killed and subunit vaccines. These vaccines consist of inactivated whole bacteria or viruses, or their purified components. These vaccines are derived from pathogenic viruses or bacteria which have been inactivated by physical or chemical processing, and either the whole microbial pathogen, or a purified component of the pathogen, is formulated as the vaccine. Vaccines of this category can be made relatively safe, through the inactivation procedure, but there is a trade-off between the extent of inactivation and the extent of the immune system reaction induced in the vaccinated patient. Too much inactivation can result in extensive changes in the conformation of immunological determinants such that subsequent immune responses to the product are not protective. This is best exemplified by clinical evaluation of inactivated measles and respiratory syncytial virus vaccines in the past, which resulted in strong antibody responses which not only failed to neutralize infectious virions, but exacerbated disease upon exposure to infectious virus. On the other extreme, if inactivating procedures are kept at a minimum to preserve immunogenicity, there is significant risk of incorporating infectious material in the vaccine formulation.

The main advantage of killed or subunit vaccines is that they can induce a significant titer of neutralizing antibodies in the vaccinated individual. Killed vaccines are generally more immunogenic than subunit vaccines, in that they elicit responses to multiple antigenic sites on the pathogen. Killed virus or subunit vaccines routinely require multiple inoculations to achieve the appropriate priming and booster responses, but the resultant immunity can be long lasting. These vaccines are particularly effective at preventing disease caused by toxin-producing bacteria, where the mode of protection is a significant titer of toxin neutralizing antibody. The antibody response can last for a significant period or rapidly rebound upon subsequent infection, due to an anamnestic or secondary response. On the other hand, these vaccines generally fail to produce a cytotoxic cellular immune response, making them less than ideal for preventing viral disease. Since cytotoxic lymphocytes are the primary vehicle for the elimination of viral infections, any vaccine strategy which cannot stimulate cytotoxic cellular immunity is usually the less preferred methodology for a virus disease, thereby resulting in attenuated virus being the usual methodology of choice.

The development of recombinant DNA technology has now made possible the heterologous production of any protein, of a microbial or viral pathogen, or part thereof, to be used as a vaccine. The vaccine constituents thus do not need to be derived from the actual pathogenic organism itself. In theory, for example, viral surface glycoproteins can be produced in eukaryotic expression systems in their native conformation for proper immunogenicity. However, in practice, recombinant viral protein constituents do not universally elicit protecting antibody responses. Further, as with killed vaccines, cellular cytotoxic immune responses are generally not seen after inoculation with a recombinant subunit protein. Thus, while this vaccine strategy offers an effective way of producing large quantities of a safe and potentially immunogenic viral or bacterial protein, such vaccines are capable of yielding only serum antibody responses and thus may not be the best choice for providing protection against viral disease.

The availability of recombinant DNA technology and the developments in immunology have led to the immunological fine mapping of the antigenic determinants of various microbial antigens. It is now theoretically possible, therefore, to develop chemically synthetic vaccines based on short peptides in which each peptide represents a distinct epitope or determinant. Progress has been made in identifying helper T-cell determinants, which are instrumental in driving B-cell or antibody immune responses. The covalent linkage of a helper T-cell peptide to a peptide representing a B-cell epitope, or antibody binding site, can dramatically increase the immunogenicity of the B-cell epitope. Unfortunately, many natural antibody binding sites on viruses are conformation-dependent, or are composed of more than one peptide chain, such that the structure of the epitope on the intact virus becomes difficult to mimic with a synthetic peptide. Thus peptide vaccines do not appear to be the best vehicle for the stimulation of neutralizing antibodies for viral pathogens. On the other hand, there is some preliminary evidence that peptides representing the determinants recognized by cytotoxic T-lymphocytes can induce CTLs, if they are targeted to the membranes of cells bearing Class I Major Histocompatibility Complex (MHC) antigens, via coupling to a lipophilic moiety. These experimental peptide vaccines appear safe and inexpensive, but have some difficulty in mimicking complex three dimensional protein structures, although there is some evidence that they can be coaxed into eliciting cytotoxic immunity in experimental animals.

Another new recombinant technique which has been proposed for vaccines is to create live recombinant vaccines representing non-pathogenic viruses, such as a vaccinia virus or adenovirus, in which a segment of the viral genome has been replaced with a gene encoding a viral antigen from a heterologous, pathogenic virus.

Research has indicated that infection of experimental animals with such a recombinant virus leads to the production of a variety of viral proteins, including the heterologous protein. The end result is usually a cytotoxic cellular immune response to the heterologous protein caused by its production after inoculation. Often a detectable antibody response is seen as well. Live recombinant viruses are, therefore, similar to attenuated viruses in their mode of action and result in immune responses, but do not exhibit the tendency to revert to a more virulent phenotype. On the other hand, the strategy is not without disadvantage in that vaccinia virus and adenovirus, though non-pathogenic, can still induce pathogenic infections at a low frequency. Thus it would not be indicated for use with immune-compromised individuals, due to the possibility of a catastrophic disseminated infection. In addition, the ability of these vaccines to induce immunity to a heterologous protein may be compromised by pre-existing immunity to the carrier virus, thus preventing a successful infection with the recombinant virus, and thereby preventing production of the heterologous protein.

In summary, all of the vaccine strategies described above possess unique advantages and disadvantages which limit their usefulness against various infectious agents. Several strategies employ non-replicating antigens. While these strategies can be used for the induction of serum antibodies which may be neutralizing, such vaccines require multiple inoculations and do not produce cytotoxic immunity. For viral diseases, attenuated viruses are regarded as the most effective, due to their ability to produce potent cytotoxic immunity and lasting immunological memory. However, safe attenuated vaccines cannot be developed for all viral pathogens.

It is therefore desirable that vaccines be developed which are capable of producing cytotoxic immunity, immunological memory, and humoral (circulating) antibodies, without having any unacceptable risk of pathogenicity, or mutation, or recombination of the virus in the vaccinated individual.

### SUMMARY OF THE INVENTION

The present invention is summarized in that an animal is vaccinated against a virus by a genetic vaccination method including the steps of preparing copies of a foreign genetic construction including a promoter operative in cells of the animal and a protein coding region coding for a determinant produced by the virus, and delivering the foreign genetic construction into the epidermis of the animal using a particle acceleration device.

The present invention is also summarized in that a genetic vaccine for the human immunodeficiency virus (HIV) is created by joining a DNA sequence encoding several or all of the open reading frames of the viral genome, but not the long terminal repeats or primer binding site, to a promoter effective in human cells to make a genetic vaccine and then transducing the genetic vaccine into cells of an individual by a particle-mediated transfection process.

The present invention is further summarized in that a genetic vaccine for influenza viruses is created by joining a DNA sequence encoding an influenza hemagglutinin-encoding gene to a promoter effective in vertebrate cells to make a genetic vaccine and then transducing the genetic vaccine into cells of an individual by a particle-mediated transfection process.

It is an object of the present invention to enable the induction of a cytotoxic immune response in a vaccinated individual to a virus through the use of a genetic vaccine.

It is a feature of the present invention in that it is adapted to either epidermal or mucosal delivery of the genetic vaccine or delivery into peripheral blood cells, and thus may be used to induce humoral, cell-mediated, and secretory immune responses in the treated individual.

It is an advantage of the genetic vaccination method of the present invention in that it is inherently safe, is not painful to administer, and should not result in adverse consequences to vaccinated individuals.

Other objects, advantages and features of the present invention will become apparent from the following specification.

### BRIEF DESCRIPTION OF THE DRAWING

FIG. 1 is a plasmid map, showing genes and restriction sites, of the plasmid pWRG1602.

FIG. 2 is a plasmid map of the genetic vaccine plasmid pCHIVpAL.

FIG. 3 depicts schematic maps of expression vectors pCMV/H1 and pCMV/control.

FIG. 4 is a graphical illustration of some of the results from one of the examples below.

FIG. 5 is a graphical illustration of the results from another of the examples below.

### DESCRIPTION OF THE PREFERRED EMBODIMENT

The method described here enables the creation of an immune response to a protein antigen by delivery of a viral gene encoding the antigenic protein into the epidermis of a patient. The epidermis has now been identified as a highly advantageous target site for such a technique. The present invention is also intended to create genetic vaccines for viral pathogens by transfecting epidermal cells of the animal to be immunized with a gene sequence capable of causing expression in the animal cells of a portion of an antigenically-intact pathogen protein, the gene sequence not including elements of the pathogen genome necessary for replication or pathogenesis.

DNA immunization, also referred to as genetic immunization, offers a new approach for realizing the advantages of an attenuated, live, or recombinant virus vaccine by mimicking the de novo antigen production and MHC class I-restricted antigen presentation obtainable with live vaccines, without the risks of pathogenic infection in either healthy or immune-compromised individuals which are otherwise associated with the use of infectious agents. DNA immunization involves administering an antigen-encoding expression vector(s) in vivo to induce the production of a correctly folded antigen(s) within the target cells. The introduction of the genetic vaccine will cause to be expressed within those cells the structural protein determinants associated with the pathogen protein or proteins. The processed structural proteins will be displayed on the cellular surface of the transfected cells in conjunction with the Major Histocompatibility Complex (MHC) antigens of the normal cell. The display of these antigenic determinants in association with the MHC antigens is intended to elicit the proliferation of cytotoxic T-lymphocyte clones specific to the determinants. Furthermore, the structural proteins released by the expressing transfected cells can also be picked up by antigen-presenting cells to trigger systemic humoral antibody responses.

For several reasons, the genetic vaccine approach of the present invention is particularly advantageously used for vaccination against immunodeficiency viruses, such as human immunodeficiency virus (HIV) and related animal viruses, simian immunodeficiency virus (SIV) and feline immunodeficiency virus (FIV). The HIV virus does not lend itself to attenuated vaccine approaches due to the inherent possibility of reversion of mutated forms of this virus. While viral protein subunit vaccines for these viruses are under development, such subunit vaccines cannot produce a cytotoxic response, which may be necessary to prevent the establishment of HIV infection or HIV-related disease. In contrast, the use of a genetic vaccine transfection strategy as described here would trigger a cytotoxic response. Also, this genetic vaccine approach allows for delivery to mucosal tissues which may aid in conferring resistance to viral introduction. HIV, for instance, is known to sometimes readily enter the body through mucosal membranes.

Another exemplary virus against which the present technique may be used is the influenza virus. The influenza virus, in its many variants, is a prevalent viral disease in mammals and birds. Because the influenza virus has been much studied, much genetic characterization of the virus exists and genetic sequences and clones of the genome of the virus and many variants of its major antigenic determinants are available.

In order to achieve the immune response sought in the vaccination process of the present invention, a genetic vaccine construction must be created which is capable of causing transfected cells of the vaccinated individual to express one or more major viral ahtigenic determinants. This can be done by identifying the regions of the viral genome that encode the various viral proteins, creating a synthetic coding sequence for one or more such proteins, and joining such coding sequences to promoters capable of expressing the sequences in mammalian cells. Alternatively, the viral genome itself, or parts of the genome, can be used. For a retrovirus, the coding sequence can be made from the DNA form of the viral genome, which, when integrated into a chromosome is referred to as the provirus, as long as the provirus clone has been altered so as to remove from it the sequences necessary for viral replication in infectious processes such as the long terminal repeats and the primer binding site. Such a provirus clone would inherently have the mRNA processing sequences necessary to cause expression of most or all of the viral structural proteins in transfected cells.

The viral genetic material used must be altered to prevent the pathogenic process from beginning. The method of altering the virus will vary from virus to virus. The immunodeficiency virus is a retrovirus carrying its genetic material in the form of RNA. During the normal infection process, the RNA is processed by an enzyme, referred to as reverse transcriptase, which converts the viral RNA into a DNA form which integrates as a provirus. The provirus for the human immunodeficiency virus (HIV) has a dozen or more open reading frames, all of which are translated to produce proteins during the infectious process. Some of the proteins are structural, and others are regulatory for steps in the infectious process. As it happens, all of the proteins produced from the provirus are actually produced from a single mRNA precursor which is differentially spliced to produce a variety of differently-spliced RNA products, which are translated into the various proteins expressed by the virus. Advantageously, it would be helpful if the transfected cell utilized in the vaccination process of the present invention expressed as many of the antigenic viral structural proteins as possible. Accordingly, it would be desirable to use as many portions of the influenza virus or retrovirus genome as necessary as the genetic vaccine coding sequence for this genetic vaccine, assuming only that sufficient portions are removed from the retroviral provirus so as to render it incapable of initiating a viral replication stage in a vaccinated individual.

A convenient strategy for achieving this objective with either the HIV or SIV viruses is based on the fact that the infectious viruses have important genetic elements necessary for replication of the viral genome, known as the long terminal repeat (LTR) elements and the primer binding site, and which are located at the ends of the native provirus sequence. The primer binding site is the site on the viral RNA where a tRNA recognizes the viral RNA, and binds to it to serve as a primer for the initiation of the reverse transcription process. Both the LTR elements and the primer binding site are necessary to permit reverse transcription to occur. Removing either the LTRs or the primer binding site would impede viral replication. Removing both the LTRs and the primer binding site from the DNA provirus ensures that the genetic sequence thus created is incapable of causing viral replication or the encoding of pathogenic viral particles.

A similar strategy may be employed for other viruses, such as the influenza viruses. Influenza protection by immunization is largely due to antibody mediated response. An influenza virus is a negative strand virus carrying its genetic material in the form of eight separate RNA segments transcribed and translated into ten gene products during the infectious process. Some of the proteins are structural, and others are regulatory for steps in the infectious process. In the instance of influenza, it is desirable and sufficient to express only one or a few viral proteins, without producing the whole set of viral proteins. This can be done by assembling an individual expression vector for each desired viral protein using standard recombinant techniques. A useful antigenic protein from the influenza virus is the hemagglutinin (HA) protein. For influenza virus genetic vaccines, for instance, protection can be achieved using only a gene encoding the antigenic Hemagglutinin viral envelope protein. For protection against a variety of influenza strains, a mixture of DNAs encoding HA subtypes can be used.

To properly express the viral genetic sequence in transfected cells, a promoter sequence operable in the target cells is needed. Several such promoters are known for mammalian systems which may be joined 5′, or upstream, of the coding sequence for the protein to be expressed. A downstream transcriptional terminator, or polyadenylation sequence, may also be added 3′ to the protein coding sequence.

Discussed above are two specific viral targets for genetic vaccination as described herein, but it should be understood that the method of the present invention is applicable to any virus for a mammalian or avian host which is capable of mounting an immune response. It is also specifically envisioned that a single genetic vaccination can include several DNAs encoding different antigenic determinants, from the same or different viruses. For example, for na influenza vaccine, it may be desirable to include several DNAs to include genes for several different HA subtypes or subgroups, or it may be desirable to include in a single vaccine genes for both an HA protein and an internal influenza virus protein, such as the NP protein. The vaccine preparation can also include genes from entirely different viruses as, for example, a combined genetic vaccination for influenza, chicken pox, and measles, in a single particle mediated treatment. The different genes can be combined by coating the different genes on the same carrier particles, or by mixing coated carrier particles carrying different genes for common delivery.

In the present invention, the genetic sequence is transferred into the susceptible individual by means of an accelerated particle gene transfer device. The technique of accelerated-particle gene delivery is based on the coating of genetic constructions to be delivered into cells onto extremely small carrier particles, which are designed to be small in relation to the cells sought to be transformed by the process. The coated carrier particles are then physically accelerated toward the cells to be transformed such that the carrier particles lodge in the interior of the target cells. This technique can be used either with cells in vitro or in vivo. At some frequency, the DNA which has been previously coated onto the carrier particles is expressed in the target cells. This gene expression technique has been demonstrated to work in procaryotes and eukaryotes, from bacteria and yeasts to higher plants and animals. Thus, the accelerated particle method provides a convenient methodology for delivering genes into the cells of a wide variety of tissue types, and offers the capability of delivering those genes to cells in situ and in vivo without any adverse impact or effect on the treated individual. Therefore, the accelerated particle method is also preferred in that it allows a genetic vaccine construction capable of eliciting an immune response to be directed both to a particular tissue, and to a particular cell layer in a tissue, by varying the delivery site and the force with which the particles are accelerated, respectively. This technique is thus particularly suited for delivery of genes for antigenic proteins into the epidermis.

It is also specifically envisioned that aqueous droplets containing naked DNA, including the viral genetic vaccine therein, can be delivered by suitable acceleration techniques into the tissues of the individual sought to be vaccinated. At some frequency, such “naked” DNA will be taken up in the treated tissues.

The general approach of accelerated particle gene transfection technology is described in U.S. Pat. No. 4,945,050 to Sanford. An instrument based on an improved variant of that approach is available commercially from BioRad Laboratories. An alternative approach to an accelerated particle transfection apparatus is disclosed in U.S. Pat. No. 5,015,580 which, while directed to the transfection of soybean plants, describes an apparatus which is equally adaptable for use with mammalian cells and intact whole mammals. U.S. Pat. No. 5,149,655 describes a convenient hand-held version of an accelerated particle gene delivery device. Other such devices can be based on other propulsive sources using, for example, compressed gas as a motive force.

A genetic vaccine can be delivered in a non-invasive manner to a variety of susceptible tissue types in order to achieve the desired antigenic response in the individual. Most advantageously, the genetic vaccine can be introduced into the epidermis. Such delivery, it has been found, will produce a systemic humoral immune response, a memory response, and a cytotoxic immune response. When delivering a genetic vaccine to skin cells, it was once thought desirable to remove or perforate the stratum corneum. This was accomplished by treatment with a depilatory, such as Nair. Current thought is that this step is not really necessary.

To obtain additional effectiveness from this technique, it may also be desirable that the genes be delivered to a mucosal tissue surface, in order to ensure that mucosal, humoral and cellular immune responses are produced in the vaccinated individual. It is envisioned that there are a variety of suitable delivery sites available including any number of sites on the epidermis, peripheral blood cells, i.e. lymphocytes, which could be treated in vitro and placed back into the individual, and a variety of oral, upper respiratory, and genital mucosal surfaces.

Gene gun-based DNA immunization achieves direct, intracellular delivery of expression vectors, elicits higher levels of protective immunity, and requires approximately three orders of magnitude less DNA than methods employing standard inoculation.

Moreover, gene gun delivery allows for precise control over the level and form of antigen production in a given epidermal site because intracellular DNA delivery can be controlled by systematically varying the number of particles delivered and the number of plasmid copies per particle. This precise control over the level and form of antigen production may allow for control over the nature of the resultant immune response.

The term transfected is used herein to refer to cells which have incorporated the delivered foreign genetic vaccine construction, whichever delivery technique is used. The term transfection is used in preference to the term transfection, to avoid the ambiguity inherent in the latter term, which is also used to refer to cellular changes in the process of oncogenesis.

It is herein disclosed that when inducing cellular, humoral, and protective immune responses after genetic vaccination the preferred target cells are epidermal cells, rather than cells of deeper skin layers such as the dermis. Epidermal cells are preferred recipients of genetic vaccines because they are the most accessible cells of the body and may, therefore, be immunized non-invasively. Secondly, in addition to eliciting a humoral immune response, separate research genetically immunized epidermal cells also elicit a cytotoxic immune response that is stronger than that generated in sub-epidermal cells. Thus, quite unexpectedly, the epidermis is the preferred target site for genes for antigenic proteins. Contrary to what some might think, a higher immune response is elicited by epidermal delivery than to any other tissue stratum yet tested. Delivery to epidermis also has the advantages of being less invasive and delivering to cells which are ultimately sloughed by the body.

Inasmuch as DNA immunization has proven successful in eliciting humoral, cytotoxic, and protective immune responses following gene gun-based DNA delivery to the skin and following direct injection by a variety of routes, it is also probable that DNA delivery to mucosal surfaces will result in immune responses as well. Since mucosal tissues are known entry points for certain viruses, particularly immunodeficiency viruses, mucosal tissues are a second preferred target for the genetic vaccines described herein. It has already been demonstrated that SIV p27-specific IgA responses could be observed following vaginal immunization with particulate p27 antigens coupled to the cholera toxin B-subunit even though this method is not compatible with the ability to elicit either CTLs or immune responses to conformational epitopes. The demonstrated ability to elicit IgA and IgG responses via vaginal immunization in the rhesus monkey is consistent with the presence of Langerhans cells and macrophages in the stratified squamous epithelium of the vagina and vaginal submucosa, respectively. Thus, it is likely that targeted DNA immunization of the vaginal and rectal mucosa surfaces will result in CTL responses and secretory IgA responses recognizing, for instance, conformationally intact SIV gp120.

Gene gun-based DNA delivery techniques are particularly well suited for developing protocols for genetic immunizations in monkey vaginal and rectal mucosal surfaces. The ability to penetrate deep into monkey epidermal and dermal tissues using 1-3 micron gold powder has already been established. The use of a standard veterinary speculum should render both the vaginal and rectal mucosal tissues accessible to the hand-held version of the gene gun.

Rectal and vaginal DNA immunizations of rhesus monkeys may be performed using expression vectors encoding an antigenic protein such as SIV gp120 or pseudovirions along with gold densities and DNA-to-gold ratios which prove optimal for skin delivery. It may be necessary to examine several depths of penetration as it is unlikely that the optimal penetration depth in mucosal tissue will mirror that seen in skin. Successful immunization may be monitored by measuring IgA and IgG responses in the serum and in vaginal and gut washes.

The adequacy of the pathogen vaccine expression vectors to be transfected into cells can be assessed by monitoring viral antigen production and antibody production in vivo after delivery of the genetic vaccine by particle acceleration or other method. Antigen monitoring techniques include RIA, ELISA, Western blotting, or reverse transcriptase assay. One may monitor antibody production directed against the antigen produced by the genetic vaccine using any of a number of antibody detection methods known to the art, such as ELISA, Western Blot, or neutralization assay.

The adequacy of the pathogen vaccine expression vectors to be transfected into cells can be assessed by assaying for viral antigenic production in mammalian cells in vitro. Susceptible mammalian cells of a cell type which can be maintained in culture, such as monkey COS cells, can be transfected in vitro by any of a number of cell transfection techniques, including calcium phosphate-mediated transfection, as well as accelerated particle transfection. Once the genetic vaccine expression vector is introduced into the susceptible cells, the expression of the viral antigens can then be monitored in medium supernatants of the culture of such cells by a variety of techniques including ELISA, Western blotting, or reverse transcriptase assay.

After confirmation that a given expression vector is effective in inducing the appropriate viral protein production in cultured cells in vitro, it can then be demonstrated that such a vector serves to induce similar protein production in a small animal model such as the mouse. The measurement of antigen expression and of antibody and cytotoxic cellular immune responses in mice in response to such a genetic vaccine would be an important demonstration of the concept and would justify initiating more rigorous testing in an appropriate animal challenge model.

After then confirming that a given expression vector is effective in inducing the appropriate viral protein production and immune response in a model laboratory animal such as the mouse, it then becomes necessary to determine the dosage and timing suitable to produce meaningful immune responses in an animal model for viral disease. Animals would receive several doses of the expression constructs by gene delivery techniques at a variety of tissue sites. The treated tissue sites would include, but would not be limited to, the epidermis, dermis (through the epidermis), the oral cavity and upper respiratory mucosa, gut associated lymphoid tissue, and peripheral blood cells. As stated above, epidermis is the preferred target. Various challenge techniques would be utilized, and the number and timing of doses of a genetic vaccine would be systematically varied in order to optimize any resulting immunogenic response, and to determine which dosage routines resulted in maximum response. Antibody responses in the treated individuals can be detected by any of the known techniques for recognizing antibodies to specific viral antigens, again using standard Western blot and ELISA techniques.

It is also possible to detect the cell-mediated cytotoxic response, using standard methodologies known to those of ordinary skill in immunological biology. Specifically, the presence of cytotoxic T-cells in the spleen or peripheral blood can be indicated by the presence of lytic activity, which recognizes histocompatible target cells which are themselves expressing the viral antigens from the immunodeficiency virus. Cell-mediated immunity directed against the antigen may be observed by co-cultivating responder splenocytes from vaccinated animals with stimulator splenocytes from naive syngeneic animals. Stimulator splenocytes are pretreated with mitomycin C and are coated with a antigenic epitope like that putatively produced in the vaccinated animal. Upon co-cultivation, responder splenocytes exposed to the antigen during vaccination will lyse stimulator cells bearing the antigenic epitope on their surfaces. One may determine the extent of cytotoxic lysis in the culture by pre-labeling the target epitope-coated cells with a radiolabel such as ⁵¹Cr and then measuring the extent of release of label after addition of responder splenocytes from a vaccinated animal.

While the best tissue sites for the delivery of a genetic vaccine for viral disease and the number and timing of doses must be empirically determined in an animal model and later confirmed in clinical studies, it is difficult at this point to predict the precise manner in which such a vaccine would be used in an actual human health care setting.

It is also important to consider that no single vaccine strategy may in itself be capable of inducing the variety of immunological responses necessary to either achieve prophylaxis in healthy individuals or forestall progression of disease in infected patients. Rather, a combination of approaches may demonstrate a true synergy in achieving these goals. Thus, it is conceivable that a combined vaccine approach incorporating a genetic vaccine, which mimics a true infection, and a killed- or subunit vaccine would be an attractive way to efficiently achieve cytotoxic immunity and immunological memory as well as high levels of protective antibody. Genetic vaccines should serve as a safe alternative to the use of live vaccines and could be used in a variety of immunization protocols and in combination with other vaccines to achieve the desired results.

### EXAMPLES

1. Preparation of Genetic Constructions for use as Immunogens

The genetic sequences for the human immunodeficiency virus (HIV) and simian immunodeficiency virus (SIV) have been fully determined, published, and are generally available. For example, the DNA sequence for the HIV strain designated LAV-1/BRU is found in GenBank at Accession Number K02013, and the nucleotide positions referred to below are from that sequence. Samples of both HIV and SIV are readily available to qualified experimenters through appropriate depositories in health research facilities.

An HIV genetic vaccine expression vector, designated pC-HIVpAL was constructed to include an 8266 base pair fragment derived from the proviral genome of HIV strain LAV-1/BRU. The fragment was the portion of the HIV DNA provirus sequence beginning at the Sac I site in nucleotide position 678 and ending at the Xho I site at nucleotide number 8944 (the nucleotide numbering convention used here assumes that nucleotide number 1 corresponds to the first nucleotide of the U3 region of the 5′ LTR). This designated fragment of the HIV genome contains all of the viral open reading frames, excepting only a portion which encodes the carboxyl terminus of the nef protein. This fragment, once transcribed, results in an mRNA which contains all of the splicing donor and acceptor sites necessary to effectuate the RNA splicing pathways actuated in an infected cell during the pathogenic process initiated by the HIV virus. The 8266 base pair fragment, isolated from strain LAV_{BRU}, was maintained and propagated in the plasmid vector clone pC-HIV.

This coding sequence fragment must be coupled to a promoter capable of expression in mammalian cells in order to achieve expression of the viral antigenic proteins in a susceptible cell. Once coupled to a promoter, this coding sequence fragment leads to the expression of the major open reading frames from the virus (including gag, pol, and env) and makes use of the native ribosomal frame shifting and mRNA processing pathways, in the same fashion as would be utilized by the virus itself. However, this fragment does lack certain viral genetic elements necessary for replication of the viral genome, including specifically the long terminal repeat (LTR) elements and the primer binding site. Thus the fragment is incapable of reverse transcription, thus inhibiting any potential pathogenic process from occurring with this genetic sequence.

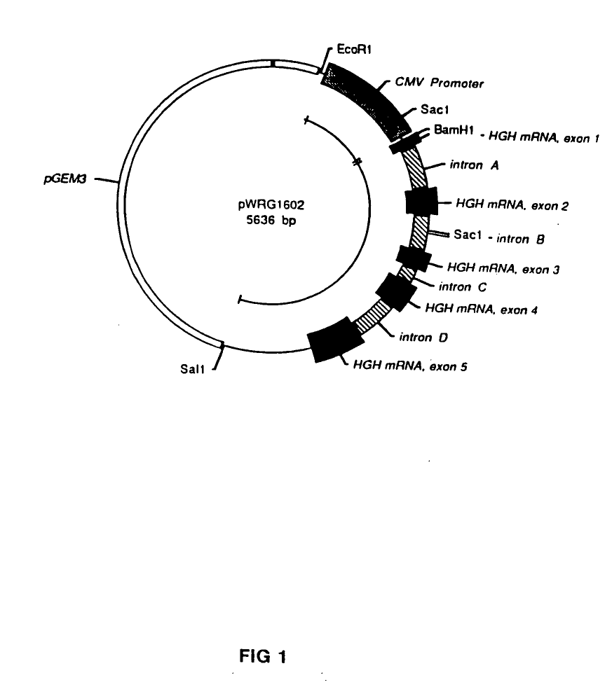

To couple the coding sequence encoding the HIV antigens to a promoter capable of expression in mammalian cells, the human cytomegalovirus (hCMV) immediate early promoter of pWRG1602 was used. In pWRG1602, illustrated by the plasmid map of FIG. 1, the hCMV immediate early promoter directs expression of a human growth hormone (hGH) gene. The hCMV promoter may be isolated from pWRG1602 on a 660 base pair Eco R1 and Bam HI restriction fragment that also contains several synthetic restriction sites added to the end of a 619 base pair immediate early promoter region.

The transcription termination segment utilized was a polyadenylation sequence from the SV40 virus. The SV40 polyadenylation fragment is an approximately 800 base pair fragment (obtained by Bgl II and Bam HI digestion) derived from the plasmid pSV2dhfr, which was formerly commercially available from the Bethesda Research Labs, catalog number 5369SS. The same polyadenylation fragment is also described in Subramani, et al., Mol. Cell. Biol., 1:854-864 (1981). This fragment also contains a small SV40 intervening sequence near the Bgl II end, with the SV40 polyadenylation region lying toward the Bam HI end of the fragment.

pC-HIVpAL was constructed in the following manner from the above-identified components. The 800 base pair SV40 fragment from pSV2dhfr was treated with Klenow DNA polymerase to “fill in” the overhanging termini. In parallel, a quantity of Bluescript M13SK(+) DNA was cleaved with Xho I (Accession Number X52325, with the Xho I site at position 668) and similarly treated with Klenow DNA polymerase. The two fragments were ligated resulting in a plasmid designated pBSpAL. The orientation of the SV40 fragment in the Bluescript vector was such that the Bam HI end, or the end containing the polyadenylation site, was oriented toward the main body of the polylinker contained in the plasmid.

Quantities of the plasmid pBSpAL were then digested with the restriction enzymes Sac I and Sal I to create a plasmid having compatible ends for ligation to a fragment created by Xho I and Sac I digestion. To this plasmid was ligated the 8266 base pair SacI/XhoI fragment from the HIV provirus, resulting in plasmid pHIVpAL, which now contains the HIV antigenic determinants coding region followed by the SV40 polyadenylation signal.

Then, the pHIVpAL plasmid was cleaved with the restriction enzyme Sac I, and the 3′ overhanging ends were deleted using Klenow DNA polymerase. Into the opening thus created, the 660 base pair fragment containing the hCMV promoter (the ends of which had been filled with Klenow DNA polymerase) was inserted.

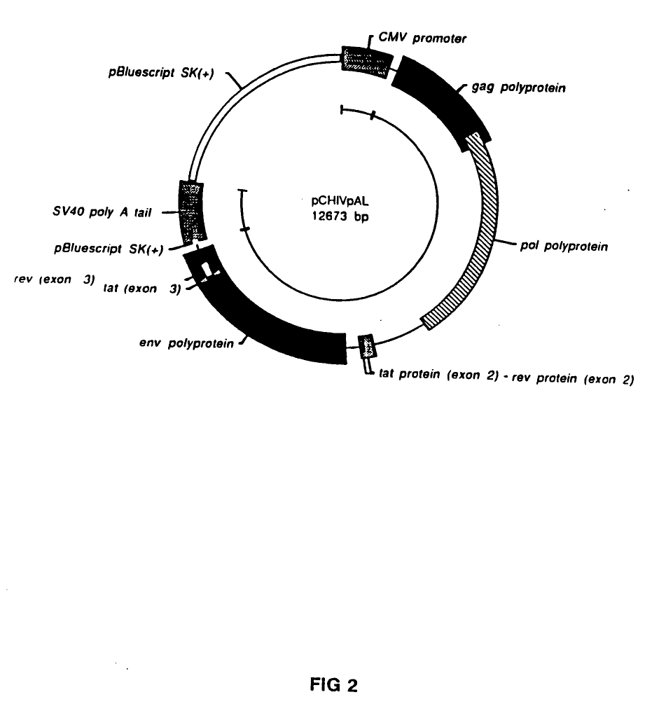

The result is the plasmid designated in FIG. 2 which contains, oriented 5′ to 3′, the hCMV immediate early promoter, the 8266 base pair fragment from the HIV genome encoding all the important open reading frames on the virus, and the SV40 polyadenylation fragment. This construct served as an HIV genetic vaccine construction for the method of the present invention.

The SIV expression vector was constructed in a manner analogous to the HIV expression vector except utilizing, in lieu of the HIV gene sequence, an 8404 base pair fragment from the proviral genome of SIVmac239 (found in GenBank at Accession No. M33262), beginning at the Nar I site at nucleotide position 823 and ending at the Sac I site at nucleotide position 9226 (following the same nucleotide numbering convention as with the HIV). The SIV genome expression fragment can be substituted for the HIV genome fragment plasmid pC-HIVpAL above.

A genetic construction, pCMV/H1, containing influenza hemagglutinin (HA) glycoprotein (subtype H1), and an appropriate control vector, pCMV/control, were prepared using standard recombinant DNA techniques. The HA glycoprotein mediates adsorption and penetration of influenza virus into animal cells and represents a major target for neutralizing antibody.

The parent vector of the pCMV vectors described herein was pBC12/CMV/IL2 expression vector. Cullen, B. R., 46 Cell 973-982 (1986). The vector backbone included, from 5′ to 3′, an SV40 origin of replication (Ori), a cytomegalovirus (CMV) immediate early promoter, an open reading frame encoding IL2, and a terminator portion of the rat preproinsulin II (RPII) gene. The RPII gene sequences included an intron and a polyadenylation site.

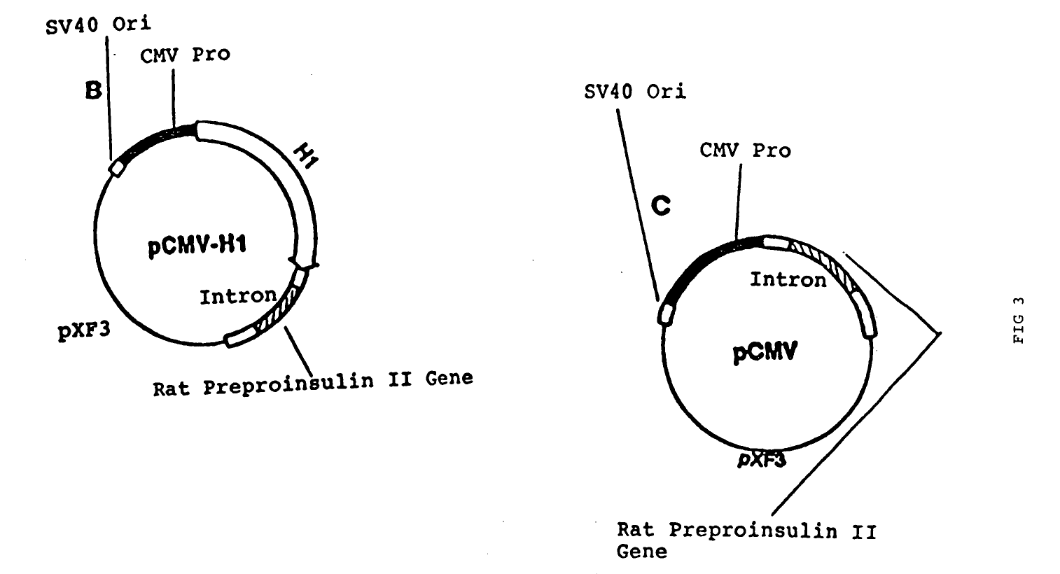

To form the vector pCMV/H1, shown at the left side of FIG. 3, a gene sequence of approximately 1.7 kbp from A/PR/8/34 (H1N1) influenza virus (Winter, G., et al., 292 Nature 72-75 (1981)) was inserted by blunt end ligation between the CMV promoter and the RPII terminator of pBC12/CMV/IL2, thereby replacing the IL2 coding region of the parent vector. Restriction endonuclease digestion analysis was used to select a clone having the viral gene inserted in the proper orientation to be expressed from the CMV promoter. The A/PR/8/34 (H1N1) gene used to engineer this vector encodes a subtype 1 hemagglutinin molecule, referred to hereinafter as H1.

The control vector, pCMV/control, at FIG. 3 right, was engineered from pBC12/CMV/IL2 by deleting the approximately 0.7 kbp DNA fragment that encodes the IL-2 gene.

2. Introduction of Genetic Vaccine into Cells in Culture

To verify the ability of the HIV genetic construct to express the proper antigenic proteins in mammalian cells, an in vitro test was conducted. Quantities of the plasmid pC-HIVpAL of FIG. 2 were reproduced in vitro. Copies of the DNA of this plasmid were then coated onto gold carrier particles before transfection into cells in culture. This was done by mixing 10 milligrams of precipitated gold powder (0.95 micron average diameter) with 50 microliters of 0.1 M spermidine and 25 micrograms of DNA of the plasmid pC-HIVpAL. The mixture was incubated at room temperature for 10 minutes. Then, 50 microliters of the 2.5 M CaCl₂ was added to the mixture, while continuously agitating, after which the sample was incubated an additional 3 minutes at room temperature to permit precipitation of the DNA onto the carrier particles. The mixture was centrifuged for 30 seconds in a microcentrifuge to concentrate the carrier particles with the DNA thereon, after which the carrier particles were washed gently with ethanol and resuspended in 10 milliliters of ethanol in a glass capped vial. The resuspension of the carrier particles in the ethanol was aided by immersion of the vial in a sonicating water bath for several seconds.

The DNA-coated carrier particles were then layered onto 35 millimeter square mylar sheets (1.7 cm on each side) at a rate of 170 microliters of DNA-coated gold carrier particles per mylar sheet. This was done by applying the ethanol suspension of the carrier particles onto the carrier sheet and then allowing the ethanol to evaporate. The DNA-coated gold particles on each mylar sheet were then placed in an accelerated particle transfection apparatus of the type described in U.S. Pat. No. 5,015,580, which utilizes an adjustable electric spark discharge to accelerate the carrier particle at the target cells to be transfected by the carrier DNA.

Meanwhile, a culture of monkey COS-7 cells had been prepared in a 3.5 cm culture dish. The medium was temporarily removed from the COS cells, and the culture dish was inverted to serve as the target surface for the accelerated particle transfection process. A spark discharge of 8 kilovolts was utilized in the process described in more detail in the above-identified U.S. Pat. No. 5,015,580. After the particle injection into the cells, two milliliters of fresh medium was added to the culture dish to facilitate continued viability of the cells.

Twenty-four hours following the accelerated particle delivery, the medium was harvested from 35 culture dishes of the cells and concentrated to test for high molecular weight HIV antigens. The medium was concentrated by high speed centrifugation. 0.5 milliliters of the unconcentrated medium was set aside for future determination of HIV p24 (a viral qaq protein) content, using a commercial ELISA kit. Fresh growth medium was added to the plates to allow continued monitoring of HIV antigen production. The harvested medium was cleared of cellular debris by first centrifuging it 1500 RPM in a standard laboratory centrifuge, and then filtering it through a 0.45 micron membrane. The cleared filtrate was layered gently onto 1 milliliter cushions of 20% glycerol, 100 mM KCl, 50 mM Tris-HCl, pH 7.8 in each of 6 ultracentrifuge tubes (Beckman SW41 rotor). The samples were centrifuged at 35,000 RPM for 70 minutes at 4° C. Following centrifugation, the medium was discarded, and the pellets were resuspended in a total of 20 microliters as 0.15 M NaCl, 50 mM Tris-HCl, pH 7.5, 1 mM EDTA.

The concentrated sample was subjected to an electrophoresis process in a pre-cast 12% SDS-polyacrylamide gel (Bio-Rad). The gel was electroblotted onto a nitrocellulose sheet using a Buchler semi-dry blotter (Model Number 433-2900) according to the manufacturer's directions. Assays were then conducted to detect the HIV viral antigens immobilized on the nitrocellulose sheet. The HIV antigens p24 and gp120 (a cleavage product of gp160, encoded by pC-HIVpAL) were detected using a Bio-Rad immunoblot assay kit (Catalog number 170-6451). To utilize the immunoblot assay kit, specific antibodies for the antigens sought to be detected are required. For use in the HIV specific assay, the following monoclonal antibodies, which are commercially available, were used. For gp120, the monoclonal antibodies were number 1001 from American Bio-Technologies and NEA 9305 from DuPont. For the antigen p24, monoclonal antibody number 4001 from American Bio-Technologies and antibody NEA 9283 from DuPont were utilized. The immunoblot assay was performed according to the manufacturer's directions, except for the direct substitution of Carnation non-fat dry milk for gelatin in all solutions calling for gelatin. The developed immunoblot assay revealed bands corresponding to both the gp120 and p24 antigens produced in the sample from the treated COS cells. A negative control produced no such bands and positive controls consisting of the antigenic proteins themselves produced bands similar to those from the samples from the treated cells. This confirmed the activity, and expression, of the plasmid pC-HIVpAL in the COS cells, and also confirmed that the molecular weight forms of the antigens were similar to those produced in the normal host cells for the virus. A parallel sample derived from non-treated COS cells showed no evidence of reactivity. There was a slight difference in mobility between the gp120 band derived from the COS cells, and the gp120 band in the positive control, which was believed to be due to differences in glycosylation.

To further demonstrate the production of HIV determinants in monkey COS cells, growth medium from treated cells was analyzed using a Coulter HIVp24 antigen assay kit (Catalog number 6603698). Samples of growth medium from the first three 24 hour periods following gene delivery were assayed for p24 antigen content, and showed to contain 42, 30, and 12 nanograms per milliliter respectively of the p24 antigen. These values reflect the amount of p24 antigen released into the medium during each 24 hour period, since the growth medium for the cells was changed completely each day following treatment. Parallel samples from non-treated COS cells exhibited no reactivity.

The ability of vector pCMV/H1 to express influenza viral hemagglutinin transiently in animal cells was confirmed by indirect immunofluorescent staining of COS cells into which the vector had been transfected. No protein product was observed from pCMV/control under the direction of the CMV promoter.

3. Detection of Viral Antigen in the Skin of Intact Mice

An accelerated particle transfection protocol was then used to deliver the plasmid pC-HIVpAL into the skin of intact whole mice. It has previously been demonstrated that accelerated particles may be utilized to deliver genes into the epidermis or dermal layer of intact animals and that the genes will express once delivered. Copies of the plasmid pC-HIVPAL were coated onto gold carrier particles, as described in the prior example, except that five micrograms of the plasmid was used per milligram of the gold carrier particles and the preparation was suspended in ethanol at a concentration of 5 mg of carrier particle per ml of ethanol. One hundred sixty-three microliters of this suspension was loaded onto each of two carrier sheets, for use in a particle acceleration protocol into intact whole mice. Two additional suspensions of the gold carrier particles were prepared as controls. The first control preparation utilized a plasmid containing the human growth hormone gene, and the second was prepared without the addition of any DNA onto the gold carrier particles.

Six BALB/c mice were anesthetized with 50 microliters of a 10:2 mixture of Ketamine/Rompin, and the abdominal hairs of the mice were shaved with clippers. Hair follicles were removed with a depilatory cream (Nair). The anesthetized mice were suspended 15 millimeters above the retaining screen on a particle delivery chamber using a plastic petri dish as a spacer, using the method described in published PCT application No. WO 91/19781. A square hole was cut in the bottom of the petri dish to allow the accelerated carrier particles to access the abdominal skin layer of the anesthetized mice. The six mice were divided into three sets of two mice each. The mice in each of the three sets received a single “blast” of carrier particles, which were accelerated utilizing an electric discharge voltage of 25 kV. The mice in each of the three sets received treatments representing the pC-HIVPAL, the growth hormone plasmid, or the gold carrier particles free of DNA, respectively.

Three days following treatment, the target skin areas were excised from the treated mice, as well as from the two control mice which had not been subjected to any particle-mediated transfection protocols. The tissue samples were minced with dissecting scissors in 600 microliters of phosphate buffered saline containing 0.5% Triton X-100. The tissue suspensions were then centrifuged at 5,000 RPM for five minutes and the resulting supernatants were collected. The supernatant samples were diluted ten-fold and analyzed for HIV p24 antigen content using the Coulter HIV p24 antigen ELISA kit (catalog number 6603698) utilizing the directions of the manufacturer.

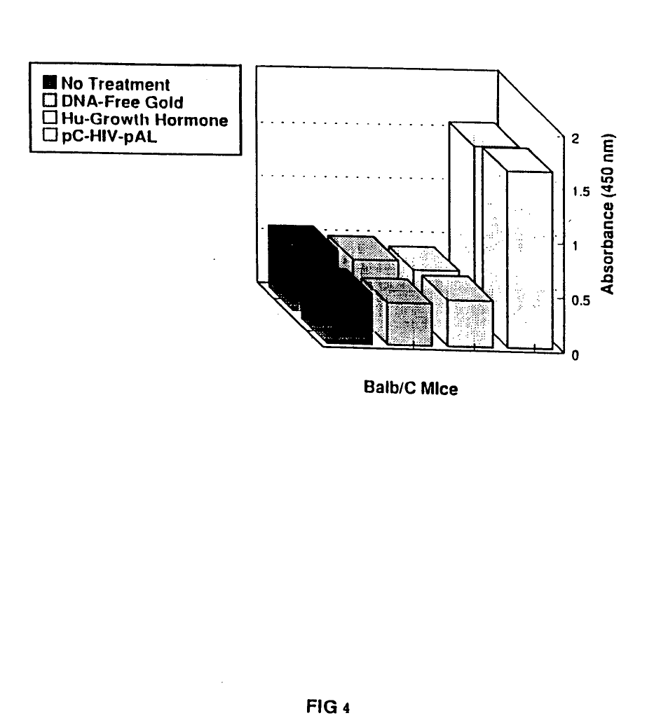

FIG. 4 illustrates the results of this protocol. Tissue samples from the pC-HIVpAL treated mice exhibited 3-fold more reactivity than the control samples, indicating that the treated tissues were synthesizing HIV p24 antigen as a result of the gene delivery protocol. After subtraction of background, this level of reactivity is consistent with the release of 0.6 nanograms of HIV p24 antigen from the minced tissue when compared to a standard curve generated with positive control reagents in the ELISA kit.

4. Detection of Serum IgG Antibodies Specific to HIV p24 in Vaccinated Mice

The next experiment was conducted to test the ability of mice to exhibit a systemic immune response to foreign proteins expressed as a result of gene delivery into epidermal cells of the mice. Copies of the plasmid pC-HIVPAL were created and coated onto gold carrier particles as described in Examples 1 and 2 above, except that 10 micrograms of plasmid DNA was used per milligram of the gold carrier particles. As a control, a heterologous plasmid containing the human growth hormone gene was also used for preparing plasmid-coated gold carrier particles for in vivo gene delivery. For this example, 5.0 micrograms of DNA was used per milligram of gold carrier particles due, to the smaller size of human growth hormone plasmid, and so as to have approximately the same number of copies of the plasmid delivered to the cells in vivo.

Ten male BALB/c mice (5 to 7 weeks old) were divided into three groups of four, three, and three mice, respectively. A first step of priming immunization was conducted on the four mice in group 1, in which each received a single treatment of accelerated particles coated only with the growth hormone plasmid. The acceleration was conducted at 25 kV by the method as described in Example 3 above. The three mice in group 2 each received a single treatment of gold carrier particles coated with the plasmid pC-HIVpAL. The mice in group 3 each received three abdominal treatments of accelerated particles which were coated with pC-HIVpAL. In the case of group 3, the blast areas were arranged so as not to be overlapping. The blasting routine for all three of the groups was repeated four and seven weeks later in order to boost the immune responses. Eight to ten days following the last treatment, retro-orbital blood samples were taken from each mouse and allowed to coagulate at 4° C. in microtainer tubes. Following centrifugation at 5000 RPM, the serum was collected.

An assay was next conducted to detect HIV p24-specific antibodies in the mouse serum by an enzyme immunoassay. This assay was performed by adsorbing 0.4 micrograms of recombinant HIV p24 antigen (American Bio-Technologies, Inc.) to each well of a 96 well microtiter plate in 50 microliter Dulbecco's phosphate buffered saline (D-PBS) by incubating overnight at 4° C. Following adsorption of the antigen, the remaining protein binding sites were blocked by the addition of D-PBS containing 2% Carnation non-fat dry milk (200 microliters per well) for two hours. The wells were then washed three times with 300 microliters D-PBS containing 0.025% Tween-20. The serum samples of 5 microliters each were diluted 1:10 with D-PBS (45 microliters), and added to a single well following which they were incubated at room temperature for one hour. After washing with D-PBS Tween-20 as described above, the presence of bound mouse antibody was detected using a goat-anti-mouse alkaline phosphatase conjugated second antibody (Bio-Rad, catalog number 172-1015) diluted 1:1500 in D-PBS-Tween-20 (50 microliters per well). After incubation for 30 minutes at room temperature, the wells were washed again and the conjugated antibody was detected using a Bio-Rad alkaline phosphatase substrate kit (Catalog No. 172-1063) according to the manufacturer's instructions. The ELISA plate was analyzed on a microplate reader using a 405 nm filter.

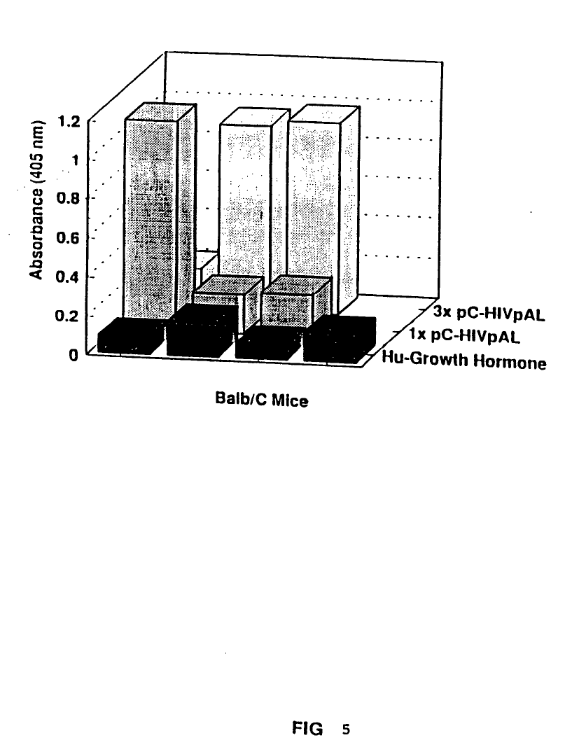

The results of this assay are illustrated in FIG. 5. One of the mice from the group which received single blasts of the pC-HIVpAL coated gold particles and two mice from the group which received three blasts of the same particles exhibited significant p24-specific antibody responses (5 to 10 fold above background). All of the sera from the control animals exhibited typical background ELISA reactivity. This example demonstrates the feasibility of inducing antigen-specific antibody responses following epidermal delivery of antigen-encoded genes coated on carrier particles into cells in an intact animal in vivo.

Thus it is demonstrated that circulating levels of antibodies to an immunodeficiency virus antigen can be created in vivo by delivering into the patient not quantities of the antigenic proteins of the virus, or the virus itself, but rather by instead delivering into the patient to be treated gene sequences causing expression of the antigenic proteins in cells in the vaccinated individual. This method thus enables the creation of a serum antibody response in vaccinated individuals without the necessity for delivering into the individual either any portions of live virus or any portions of the genetic material which are capable of effectuating replication of the virus in individuals.

5. Influenza Virus Immunization

Using the protocols detailed below, the above-described influenza virus DNA genetic constructions were transferred into groups of six- to eight-week old BALB/c mice which were then lethally challenged with mouse-adapted A/PR/8/34 (H1N1) influenza virus. The H1 gene of A/PR/8/34 virus is identical to the H1 gene of the mouse genetic immunization vector pCMV/H1.

Plasmids pCMV/H1 and pCMV/control were prepared separately for genetic immunization as described above. Various amounts of plasmid DNA were mixed with 10 mg of 0.95 micron gold powder (Degussa, South Plainfield, N.J.) in a 1.5 ml microcentrifuge tube containing 50 μl of 0.1M spermidine. Plasmid DNA and gold particles were co-precipitated by adding 50 μl of 2.5 M CaCl₂ while vortexing. The precipitate was allowed to settle and was washed with absolute ethanol and resuspended in 2.0 ml of ethanol. The gold/DNA suspension was transferred to a capped glass vial and immersed in a sonicating water bath for 2-5 seconds to resolve clumps. The gold/DNA suspension (163 μl) was layered onto mylar sheets (1.8 cm×1.8 cm) and allowed to settle for several minutes. The meniscus was then broken and excess ethanol was removed by aspiration. Gold/DNA-coated mylar sheets were dried and stored under vacuum. While the amount of gold per sheet was constant, the amount of DNA per sheet ranged from 0.2 μg to 0.0002 μg.

Mice were anesthetized with 30 μl of Ketaset:Rompun (10:2). Abdominal target areas were shaved and treated with depilatory (Nair) for 2 minutes to remove residual stubble and stratum corneum. Target areas were thoroughly rinsed with water prior to gene delivery. DNA-coated gold particles were delivered into the abdominal epidermis using an Accell particle-acceleration instrument (as described in U.S. Pat. No. 5,149,655) which employs an electric spark discharge as the motive force. A discharge voltage of 17 kV is preferred, as delivery of gold particles into epidermal tissue at that voltage causes no visible cellular injury yet results in strong intracellular expression of transferred genes. Two non-overlapping DNA deliveries were performed on each mouse, the first at time 0 and the second four weeks later.

For comparison, pCMV/H1 and pCMV/control were separately inoculated into six- to eight-week old BALB/c mice by intravenous (tail vein, iv), intraperitoneal (ip), intramuscular (quadriceps, im), intranasal (DNA drops administered to mice anesthetized with Metofane, m), intradermal (footpad, id), or subcutaneous (scruff of the neck, sc) delivery. DNA used for inoculation was first diluted at 100 μg per 100 μl in saline. Two inoculations were given to each mouse, at time 0 and at 4 weeks.

Ten days after the second DNA treatment (particle acceleration or inoculation), each mouse was anesthetized with Metofane (Pitman-Moore, Mundelein, Ill.) and challenged with mouse adapted A/PR/8/34 (H1N1) influenza virus in 100 μl of saline supplemented with 0.1% bovine serum albumin (BSA). The challenge dose was empirically chosen to give 100% death in naive mice 1.5 to 2 weeks post-challenge.

Table 1 demonstrates that delivery into mouse skin of just 0.4 μg of a genetic construction encoding an influenza hemagglutinin gene afforded complete protection against challenge by a lethal dose of influenza virus. Even administration of tenfold less DNA by particle acceleration resulted in greater than 50% survival rates, with only transient influenza symptoms, after challenge. In contrast, intramuscular, intravenous, intranasal, intradermal, or subcutaneous delivery required 50-300 μg of DNA to achieve survival rates of 67% to 95%. The DNA inoculation data presented herein were pooled from 4 independent trials for the injection of DNA in saline and from four independent trials for particle mediated DNA delivery.

All survivors developed influenza symptoms, with the severity of disease being inversely correlated with survival. Typically, survival was highest in those groups that received the most DNA by any protocol. However, the absolute amounts of DNA required for survival after particle-acceleration-mediated genetic immunization were markedly lower than any other. The data presented demonstrate that genetic immunization in mice may be accomplished using at least 200-fold less DNA in a non-invasive particle acceleration protocol than in the injection protocols described.

TABLE 1

| | Route of | Dose | Signs of | Survivors/ | % |
|---|---|---|---|---|---|
| DNA | inoculation | (ug) | influenza | tested | survival |
| pCMV/H1 | iv, ip, im | 300 | ++ | 21/22 | 95% |
| in saline | im | 200 | ++ | 18/19 | 95% |
| | iv | 100 | ++ | 10/12 | 83% |
| | in | 100 | +++ | 13/17 | 76% |
| | id | 50 | 9/12 | 75% | |
| | sc | 100 | 4/6 | 67% | |
| | ip | 100 | 0/6 | 0% | |
| pCMV/ | various | 0-300 | 3/24 | 13% | |
| control | | | | | |
| in saline | | | | | |
| pCMV/H1 | ed | 0.4 | none | 21/22 | 95% |
| on gold | ed | 0.04 | +++ | 7/11 | 64% |
| beads | ed | 0.004 | +++++ | 0/5 | 0% |
| | ed | 0.0004 | +++++ | 0/4 | 0% |
| pCMV/ | ed | 0.4 | +++++ | 3/22 | 14% |
| control on | | | | | |
| gold beads | | | | | |

On the table above and those below, the + result indicates that the animal had transient weight loss with maintenance of normal fur and activity; the ++ result indicates that the animals had transient weight loss with some ruffling of fur and lethargy; the +++ result indicates transient weight loss with more severe ruffling of fur and lethargy; the ++++ result indicates more prolonged weight loss coupled with severe fur ruffling and lethargy; the +++++ result indicates weight loss and severe signs of influenza leading to death. iv=intravenous, ip=intraperitoneal, im=intramuscular, in=intranasal, sc=sub cutaneous, ed=epidermal.

A striking observation from the above data comparing gene-gun delivery to saline delivery of DNA vaccine is that the delivery by particle acceleration was strikingly more efficient. The delivery by accelerated particle achieved protection with 250-2500 times less DNA compared to saline injection delivery. The ability of the particle acceleration device to target epidermis is advantageous since it appears that DNA-expressed antigens are efficiently detected by skin associated lymphoid tissue.

6. Anti-HA Antibody Titers After Genetic Immunization

Sera were obtained from post-challenge mice that had previously been either inoculated with pCMV/H1 DNA or pCMV/control in saline or genetically immunized with pCMV/H1 DNA by particle acceleration. These sera were examined for the presence of anti-HA antibody using hemagglutinin inhibition (HI) tests performed in microtiter plates as described by Palmer, D. F., et al., in Advanced Laboratory Techniques for Influenza Diagnosis, Immunology Series, No. 6, pp. 51-52, U.S. Department of Health, Education and Welfare, Washington, D.C. (1975). Background activity was removed from the mouse sera by pretreatment with kaolin.

DNA vaccinations by the various routes appeared to prime antibody responses. Antibody responses were assayed using tests for hemagglutination-inhibiting activity and ELISA activity (see Table 2 following, this data also presented in Fynan et al., PNAS 90:11478-11482 hereby incorporated by reference). The DNA vaccinations and boosts raised only low to undetectable titers of hemagglutination-inhibiting antibodies and ELISA activity. These low levels of activity underwent rapid increases post challenge. Protection occurred in mice that did not have detectable levels of anti-influenza antibodies prechallenge. However, the best protection occurred in groups in which the DNA inoculations had raised detectable titers of antibody.

TABLE 2
Antibody Responses in Vaccine Trials
Testing Routes of Inoculation in Mice

| | | | | Titers of antibody to | | | |
|---|---|---|---|---|---|---|---|
| | | | | A/PR/8/34 (HIN1) | | | |
| | Time of | | No. | | ELISA value × 10⁻² | | |
| DNA and route | bleed | | tested | HI | IgM | IgG | IgA |
| pCMV/HI | | | | | | | |
| in saline | | | | | | | |
| i.v. | Prevac | | 2(12) | < | < | < | < |
| | 10 | d PB | 2(12) | < | < | 8 | 4 |
| | 4 | d PC | 1(6) | 20 | < | 128 | 4 |
| | 14-19 | d PC | 2(10) | 113 | 1 | 256 | 4 |
| i.m. | Prevac | | 3(19) | < | < | < | < |
| | 10 | d PB | 3(19) | < | < | 3 | < |
| | 4 | d PC | 2(13) | 6 | < | 32 | 2 |
| | 14-19 | d PC | 3(18) | 127 | < | 406 | 2 |
| i.n. | Prevac | | 3(17) | < | < | < | < |
| | 10 | d PB | 3(17) | < | < | 2 | 1 |
| | 4 | d PC | 2(11) | < | 1 | 2 | 1 |
| | 14-19 | d PC | 3(17) | 160 | 2 | 202 | 2 |
| pCMV/control | | | | | | | |
| in saline | | | | | | | |
| Various | Prevac | | 3(16) | < | < | < | < |
| | 10 | d PB | 3(16) | < | < | < | < |
| | 4 | d PC | 2(9) | < | < | < | < |
| | 14-19 | d PC | 1(2) | 320 | < | 256 | < |
| pCMV/HI | Prevac | | 2(10) | < | < | < | < |
| gene gun | 10 | d PB | 3(16) | 10 | 1 | 10 | < |
| | 4 | d PC | 3(16) | 20 | 2 | 64 | < |
| | 14-19 | d PC | 3(15) | 160 | < | 645 | < |
| pCMV/control | Prevac | | 2(12) | < | < | < | < |
| gene gun | 10 | d PB | 3(16) | < | 1 | < | < |
| | 4 | d PC | 3(16) | < | 2 | < | < |
| | 14-19 | d PC | 1(3) | NT | 4 | 512 | < |

Use of ELISAs to score the isotypes of the anti-influenza virus antibodies demonstrated that the immunizations had primed IgG responses. Low titers of anti-influenza IgG could be detected in the sera of mice vaccinated by gun delivery, iv, or iminoculations of DNA. Borderline to undetectable titers of IgG were present in the sera of mice receiving DNA nose drops (consistent with the poorer protection provided by this route of DNA administration). By 4-days post challenge, increased levels of IgG were detected in mice undergoing the best protection. By contrast, mice receiving control DNA did not have detectable levels of anti-influenza virus IgG until the second serum collection post challenge. This was consistent with vaccinated, but not control groups, undergoing a secondary antibody response to the challenge.

7. Use of PCMV/H1 DNA Transcriptional Unit to Protect Ferrets Against A/PR/8/34 (H1N1) Influenza Challenae

Studies of pCMV/H1 DNA immunization in a ferret model were undertaken because this influenza model has many similarities to human influenza infections. In the initial experiment, ferrets were immunized with purified pCMV/H1 DNA in saline by intramuscular inoculations at a one month interval. Young adult female ferrets were prebled and vaccinated with 500 μg of pCMV/H1 or pCMV/control DNA in saline by two injections of 125 μl in each hind leg for a total inoculation volume of 500 μl. One ferret received three intramuscular inoculations of 500 μg of pCMV/H1 DNA at one month intervals while a second animal received two intramuscular inoculations of 500 μg of DNA at one month intervals. The control animal received three 500 μg intramuscular inoculations of pCMV/control DNA at one month intervals.

Metofane-anesthetized ferrets were challenged with 10^{7.7} egg infectious doses₅₀ of A/PR/8/34 H1N1) via the nares at one week after the final DNA inoculation. Nasal washes were collected at days 3, 5 and 7 post challenge under ketamine anesthetic. Titration of virus in nasal washes was done in eggs as described (Katz, J. M. and R. G. Webster, J. Infect. Dis. 160:191-198 (1989)). The results are presented in Table 3, below.

TABLE 3
Protection of Ferrets Against an H1 Virus
by Intramuscular Inoculation of pCMV/H1 DNA

| | | | Virus Titer in NasaI | | |
|---|---|---|---|---|---|
| | | | Washes, log₁₀ egg | | |
| | No. of DNA | Ferret | infectious doses₃₀/ml | | |
| DNA | Administrations | ID No. | day 3 | day 5 | day 7 |
| pCMV/H1 | 3 | 901 | 5.5 | 1.5 | <1 |
| | 2 | 903 | 5.7 | 4.7 | <1 |
| pCMV/control | 3 | 907 | 6.5 | 6.2 | <1 |

Analysis of nasal washes revealed similar high titers of virus in the washes of all of the ferrets at 3 days post challenge. Interestingly, the ferret receiving three inoculations of pCMV/H1 had largely cleared the nasal infection by five days post challenge, with its five day nasal wash containing less than 10 egg infectious doses₅₀ of virus per ml. At this time, the ferret receiving two inoculations of pCMV/H1 DNA had a ten fold reduction in the titer of virus in its nasal wash. By contrast, the ferret receiving control DNA had modest if any reduction in the titer of virus in its nasal wash. By 7 days post challenge, all of the ferrets had cleared their nasal infections. The much more rapid clearing of virus in the ferret receiving three intramuscular inoculations of pCMV/H1 DNA and the somewhat more rapid clearing of virus in the ferret receiving two intramuscular inoculations of pCMV/H1 DNA than in the two ferrets receiving control DNA suggest that the intramuscular inoculations of pCMV/H1 had raised some anti-influenza immunity.

Gene Gun Inoculation

To increase the efficiency of the induction of immunity, a second experiment was undertaken in ferrets using the Accell accelerated particle gene delivery instrument to deliver DNA coated gold beads into the skin of ferrets. The abdominal epidermis was used as the target for particle mediated DNA with ferrets receiving two administrations of DNA at a one month interval. Particle mediated inoculations were delivered to Ketamine-anesthetized young adult female ferrets. Skin was prepared by shaving and treating with the depilatory agent NAIR (Carter-Wallace, New York). DNA beads (1 to 3 microns) were prepared for inoculations as previously described (Fynan et al., Proc. Natl. Acad. Sci. USA 90:11478-11482 (1993)). A delivery voltage of 15 kV was used for inoculations. Ferrets were inoculated with either 2 μg or 0.4 μg of DNA. Ferrets inoculated with either 2 μg of DNA received 10 shots with each shot consisting of 0.8 mg of meads coated with 0.2 μg of DNA. Ferrets receiving 0.4 μg of DNA received two of these shots.

Metofane-anesthetized ferrets were challenged at one week after the second DNA immunization by administration of 10^{6.7} egg infectious doses of A/PR/8/34 (H1N1) virus via the nares. This challenge was 10 fold lower than in the experiment using intramuscular inoculation because of the high levels of virus replication in the first challenge. Nasal washes were collected at days 3 and 5 post challenge under Ketamine anesthetic and the virus titered as described below. Data are presented in Table 4, below.

TABLE 4
Protection of Ferrets Against an H1 Virus
by Gene Gun Inoculation of pCMV/H1 DNA

| | | |
|---|---|---|
| | | Virus Titer in Nasal |
| | | Washes, log₁₀ egg |
| Amount of | Ferret | infectious dose₅₀/ml |

| DNA | DNA (μg) | ID No. | day 3 | day 7 |
|---|---|---|---|---|
| pCMV/H1 | 2 | 927 | <1 | <1 |
| | | 931 | <1 | <1 |
| | | 933 | <1 | <1 |
| | 0.4 | 926 | 4.3 | <1 |
| | | 929 | 3.9 | <1 |
| | | 933 | <1 | <1 |
| pCMV/ | 2 | 932 | 3.5 | <1 |
| control | | 934 | 3.7 | <1 |

Analysis of post-challenge nasal washes in gene gun vaccinated ferrets revealed that the three ferrets receiving 2 μg of DNA and one of the three ferrets receiving 0.4 μg of DNA were completely protected from the challenge. This was shown by the inability to recover virus in the nasal washes of these animals at 3 days post challenge. The remaining two animals receiving 0.4 μg of DNA and the control animals were not protected, with easily detected titers of virus present in the nasal washes of the animal at three days post challenge. In this experiment, all animals (control and vaccinated) had no detectable virus in their nasal washes by five days post challenge.

Ferrets from the gene gun experiment were next analyzed for antibody responses to the DNA administrations and to the challenge virus. These assays tested for neutralizing activity for A/PR/8/34 (HlNl). The titrations of antibodies were done as described (Katz, J. M. and R. G. Webster, J. Infect. Dis. 160:191-198 (1989)). Titers of neutralizing activity are the reciprocals of the highest dilution of sera giving complete neutralization of 200 50% tissue culture infectious doses of virus. Data are presented in Table 5 below.

TABLE 5
Neutralizing Antibody in Ferrets Vaccinated with
Gene Gun-Delivered pCMV/H2 DNA and Challenged with
A/Pr/8/34 (H2N1) Influenza Virus

| | | | Neutralizing Antibody | | | |
|---|---|---|---|---|---|---|
| | | | | Post-boost, | Post | Post |
| | Amount of | Ferret | Pre- | pre- | challenge | challenge |
| DNA | DNA (μg) | ID. No. | inoculation | challenge | (7 days) | (14 days) |
| pCMV/H1 | 2 | 927 | <10 | <10 | 2500 | 1800 |
| | | 931 | <10 | 800 | 2800 | 1800 |
| | | 933 | <10 | 130 | 4000 | 4000 |
| | 0.4 | 926 | <10 | <10 | 25000 | 25000 |
| | | 929 | <10 | <10 | 4000 | 1300 |
| | | 933 | <10 | <10 | 7900 | 5600 |
| pCMV/control | 2 | 932 | <10 | <10 | 5600 | 4000 |
| | | 934 | <10 | <10 | 5600 | 7900 |

Neutralizing antibody post DNA boost but prior to challenge was detected in two of the animals receiving 2 μg of gene gun-delivered DNA. No neutralizing antibody was detected in the pre-challenge sera of the third animal receiving 2 μg of DNA (an animal that was completely protected against the presence of virus in nasal washes).

Neutralizing antibody was also not detected in the sera of the ferret receiving 0.4 μg of DNA that did not develop virus in its nasal wash.

In animals with prechallenge antibody, protection was presumably due to the presence of neutralizing antibody as well as the mobilization of memory responses for neutralizing antibody. In protected animals without detectable levels of pre-challenge antibody, protection was likely due to the rapid mobilization of memory responses by the infection, with the mobilized responses controlling the infection. Protection in vaccinated animals in the absence of pre-challenge antibody has also been observed in prior DNA vaccination studies in mice and chickens (See Tables 3, 5 and 9) (Fynan et al., Proc. Natl. Acad. Sci. USA 90:11478-11482 (1993); Robinson et al., Vaccine 11:957-960 (1993)) and in vaccine trials using retrovirus and pox virus vectors to express the influenza virus hemagglutinin glycoprotein (Hunt et al., J. Virol. 62:3014-3019 (1988); Webster et al., Vaccine 9:303-308 (1991)).

1-9. (canceled)

10. A method for inducing an immune response in a mammal against influenza virus, comprising delivering carrier particles that are each coated with a plurality of constructs into mammalian epidermal cells using a particle acceleration device, wherein (a) each construct comprises a nucleic acid sequence that is (i) operably linked to a promoter that is functional in a mammalian cell and (ii) encodes an influenza virus antigen from a different influenza strain; (b) the carrier particles are small in size relative to the size of epidermal cells in the mammal; and (c) sufficient influenza antigens are expressed by the nucleic acid sequences of the constructs in the epidermal cells of the mammal to induce an influenza-specific immune response in the mammal.

11. The method of claim 10, wherein at least one of said nucleic acid sequences encodes an antigenic influenza virus protein.

12. The method of claim 11, wherein the influenza virus protein is hemagglutinin.

13. The method of claim 12, wherein the hemagglutinin is of subtype H1.

14. The method of claim 13, wherein said nucleic acid sequences encode a plurality of antigenic influenza virus proteins.

15. The method of claim 14, wherein said nucleic acid sequences encode a plurality of hemagglutinin subtypes.

16. Carrier particles for inducing an immune response in a mammal against influenza virus, wherein the carrier particles are each coated with a plurality of nucleic acid constructs as defined in claim 10 and are small in size relative to the size of mammalian epidermal cells.

17. Carrier particles of claim 16, wherein at least one influenza virus protein encoded by said constructs is hemagglutinin.

18. Carrier particles of claim 17, wherein the influenza virus protein is hemagglutinin is of subtype H1.

19. Carrier particles of claim 16, wherein at least one nucleic acid sequence in said constructs encodes an antigenic influenza virus protein.

20. Carrier particles of claim 16, which are gold particles.

21. A particle acceleration device containing a dose of the gold particles of claim 20.

22. A method for inducing an immune response in a mammal against influenza virus, comprising:
(a) preparing a plurality of different constructs where each construct (i) comprises a nucleic acid sequence that is (i) operably linked to a promoter that is functional in a mammalian cell and (ii) encodes an antigenic influenza virus hemagglutinin protein from a different influenza strain;
(b) combining the different constructs by coating each of the different constructs onto the same carrier particles, wherein the carrier particles are small in size relative to the size of epidermal cells in the mammal; and
(c) delivering the mammal by delivering the coated carrier particles into epidermal cells of the mammal using a particle acceleration device whereby the nucleic acid sequences are expressed in said epidermal cells to provide influenza antigen sufficient to induce an influenza-specific immune response in the mammal.

23. The method of claim 22, wherein said nucleic acid sequences encode antigenic influenza virus hemagglutinin proteins from a plurality of different subgroups.

24. The method of claim 22, wherein said nucleic acid sequences encode antigenic influenza virus hemagglutinin proteins from a plurality of different subtypes.

25. The method of claim 22, wherein said method induces an immune response against a variety of different influenza virus strains.

26. The method of claim 22, wherein said immune response comprises a systemic humoral immune response.

27. The method of claim 22, wherein said immune response comprises a memory response.

28. The method of claim 22, wherein said immune response comprises a cytotoxic immune response.

29. A method for preparing carrier particles for inducing an immune response in a mammal against influenza virus, comprising coating onto carrier particles a plurality of different constructs, each of which comprises a nucleic acid sequence that is (i) operably linked to a promoter that is functional in a mammalian cell and (ii) encodes an influenza virus antigen from a different influenza strain and where the carrier particles are small in size relative to the size of epidermal cells in the mammal.
